(19) United States
(12) Patent Application Publication
(10) Pub. No.: US 2011/0201037 A1
(43) Pub. Date: Aug. 18, 2011

(54) USE OF ERYTHROPOIETIN TO DEVELOP SMALL MOLECULE INHIBITORS OF JANUS KINASE-2

(76) Inventors: Eric Bachman, Swampscott, MA (US); Jan-Rung Mo, Needham, MA (US); Anjili Mathur, Revere, MA (US)

(21) Appl. No.: 12/671,528

(22) PCT Filed: Jul. 29, 2008
(86) PCT No.: PCT/US08/09133

§ 371 Date: Feb. 1, 2010

Related U.S. Application Data
(60) Provisional application No. 60/963,004, filed on Aug. 2, 2007.

(51) Int. Cl. C12Q 1/48

(52) U.S. Cl. 435/15

(57) ABSTRACT

The invention pertains to a method for identifying compounds that inhibit the erythropoietin-induced JAK2 kinase activity in vivo. The present invention provides a method for detecting small molecule JAK2 inhibitors in a rapidly created rodent model that phenocopies human PV through Epo stimulation. The method comprises the steps of (a) dosing a rodent with erythropoietin (Epo) and a test compound, (b) collecting blood samples after the dose is administered, (c) measuring phosphorylated STAT5 levels in the blood sample, and (d) determining JAK2 inhibitor levels in the blood samples.

### BACKGROUND OF THE INVENTION

The instant invention pertains to a method for identifying compounds that inhibit the erythropoietin-induced JAK2 kinase activity in vivo.

A human disease characterized by increased red blood cell mass, plethora and susceptibility to vascular thromboses has been recognized for over a century (Tefferi, et al., Semin. Hematol., 2005. 42(4): p. 206-20). Recently, this condition was termed polycythemia vera (PV), and is now grouped with other Philadelphia chromosome-negative myeloproliferative disorders (MPDS) including myeloid metaplasia with myelofibrosis (MMM) and essential thrombocythemia (ET). Patients with PV are at increased risk of vascular thromboses and hemorrhage. (Chievitz, et al., Acta. Med. Scand., 1962. 172: p. 513-23). Although periodic phlebotomy along with antiplatelet treatment (aspirin) have lowered this risk and improved survival, patients with PV continue to have debilitating morbidities and reduced lifespan (Landolfi, et al., N. Engl. J. Med., 2004. 350(2): p. 114-24; Passamonti, et al., Am. J. Med., 2004. 117(10): p. 755-61); Merup, et al., Acta Oncol., 2002. 41(1): p. 50-5; Diehn, et al., Br. J. Haematol., 2001. 115(3): p. 619-21; Vannucchi, et al., Blood, 2007). In addition, some patients develop terminal myelofibrosis or acute myeloid leukemia (Schafer, A. I., Blood, 2006. 107(11): p. 4214-22.) Thus, effective treatments for PV are warranted.

It is well known that the Erythropoietin-EpoR-JAK2-STAT5 pathway plays a critical role in the development and proliferation of erythropoietic lineages. Erythropoietin binds to a single type 1 erythropoietin receptor (EpoR), and induces a series of signaling events that result in homodimerization of EpoR, autophosphorylation of JAK2, transphosphorylation of EpoR, and activation/docking of signaling molecules including signal transducers and stimulators of transcription (STATs) (Kisseleva, et al., Gene, 2002. 285(1-2): p. 1-24; Lodish, et al., Cold Spring Harb Symp Quant Biol, 1995. 60: p. 93-104). Consequently, a series of investigations into a potential molecular basis of PV, via activation of the erythropoietin signaling pathway, were undertaken (Tefferi, Mayo Clin. Proc., 2003. 78(2): p. 174-94).

Several independent labs discovered that nearly all patients with PV harbor a single mutation (V617F) in the JH2 pseudokinase domain of JAK2. This mutation is associated with an increased level of JAK2 phosphorylation and constitutive activation by phosphorylation of Stat5 (Zhao, et al., J. Biol. Chem., 2005. 280(24): p. 22788-92; James, et al., Nature, 2005. 434(7037): p. 1144-8; Kralovics, et al., N. Engl. J. Med., 2005. 352(17): p. 1779-90; Levine, et al., Cancer Cell, 2005. 7(4): p. 387-97.) Furthermore, transplantation of V617F-transduced bone marrow cells into lethally irradiated mice results in PV that is nearly identical to human PV Wemig, et al., Blood, 2006. 107(11): p. 4274-81; Zaleskas, et al., PLoS ONE, 2006. 1: p. e18; Lacout, et al., Blood, 2006. 108(5): p. 1652-60; Bumm, et al., Cancer Res., 2006. 66(23): p. 11156-65). The discovery a single nucleotide mutation in JAK2 in most PV patients, combined with the observation that this mutation is sufficient to cause PV in mice, has generated interest in the identification of JAK2 inhibitors for the potential treatment of PV. However, the current rodent models of human PV require bone marrow transplantation of mutant JAK2 rodents to create an in vivo model, which requires a 6-10 week preparation. Wernig, et al., Blood, 2006. 107(11): p. 4274-81; Zaleskas, et al., PLoS ONE, 2006. Therefore, a rodent model that mimics human PV, but requires less time to create than the transplantation model, would be a valuable tool for identifying small molecule JAK2 inhibitors.

### SUMMARY OF THE INVENTION

The instant invention pertains to a method for identifying compounds that inhibit the erythropoietin-induced JAK2 kinase activity in vivo.

The first embodiment of the present invention is directed to a method for identifying a compound as an inhibitor of JAK2 activity comprising the steps of (a) dosing a rodent with erythropoietin (Epo) and a test compound, (b) collecting blood samples after the dose is administered, (c) measuring phosphorylated STAT5 levels in the blood sample, and (d) determining JAK2 inhibitor levels in the blood samples.

In a class of the embodiment, the Epo is selected from the group consisting of: (a) human recombinant Epo, (b) endogenous human Epo, (c) mouse Epo, and (d) rat Epo.

In a subclass of the embodiment, the Epo is human recombinant Epo.

In another embodiment of the present invention, the rodent is selected from the group consisting of rat and mouse.

In a class of the embodiment, the blood samples may be collected any time from 0 to 48 hours after dosing the rodent with the EPO and the test compound.

In a subclass of the embodiment, the blood sample may be collected at a time selected from the group consisting of (a) 1 hour, (b) 3 hours, and (c) 8 hours after dosing the rodent with the Epo and test compound.

In a class of the embodiment, the dosing of the rodent with Epo and the test compound occurs at the intervals selected from the group consisting of (a) immediately, (b) daily, and (c) intermittently.

In a class of the embodiment, the collection of blood sample is obtained through the methods selected from the group consisting of (a) retro-orbitally, (b) cardiac puncture, and (c) tail bleed.

In one embodiment, the invention provides an acute method for screening a test compound for JAK2 inhibitory activity, wherein the method comprises the steps of dosing a rodent with Epo and a test compound; collecting a blood sample from the rodent within 48 hours of dosing; determining the level of phosphorylated STAT5, thereby determining whether the compound inhibits JAK2 activity; and determining levels of the test compound in the blood, thereby determining the 1050 value of said compound.

In another embodiment, the invention provides a non-acute method for screening a test compound for JAK2 inhibitory activity, wherein the method comprises the steps of dosing a rodent with Epo and a test compound, daily for up to 28 days; collecting a blood sample from the rodent after the final dose; determining the level of phosphorylated STAT5, thereby determining whether the compound inhibits JAK2 activity; and determining levels of the test compound in the blood, thereby determining the 1050 value of said compound.

Yet, in another embodiment, the invention provides a non-acute method for screening a test compound for JAK2 inhibitory activity, wherein the method comprises the steps of dosing a rodent with Epo and a test compound intermittently for up to 28 days; collecting a blood sample from the rodent after the final dose; determining the level of phosphorylated STAT5, thereby determining whether the compound inhibits JAK2 activity; and determining levels of the test compound in the blood, thereby determining the 1050 value of said compound.

Unless otherwise defined, all technical and scientific terms used herein in their various grammatical forms have the same meaning as commonly understood by one of ordinary skill in the art to which this invention belongs. Although methods and materials similar to those described herein can be used in practice or testing of the present invention, the preferred methods and materials are described below. In case of the conflict, the present specification, including definitions, will control. In addition, the materials, methods, and examples are illustrative only and are not limiting.

Further features, object, and advantages of the present invention are apparent in the claims and the detailed description that follows. It should be understood, however, that the detailed description and the specific examples, while often indicating preferred aspects of the invention, are given by way of illustration only, since various changes and modifications within the spirit and scope of the invention will become apparent to those skilled in the art from this detailed description.

### DETAILED DESCRIPTION OF THE INVENTION

The invention pertains to a method for identifying compounds that inhibit the erythropoietin-induced JAK2 kinase activity in vivo. The present invention provides a method for detecting small molecule JAK2 inhibitors in a rapidly created rodent model that phenocopies human PV through Epo stimulation.

The discovery that nearly all patients with PV harbor a single nucleotide mutation in JAK2, and that this mutation is sufficient to cause PV in mice, has generated great interest in developing inhibitors of JAK2 for JAK2-driven diseases. The present invention provides a method for creating an Epo-driven rodent model of human PV that both phenocopies the human disease and provides a rapid model for testing JAK2 inhibitors for polycythemia. This model results in polycythemia, induction of Bcl-x in erythroid cells, splenomegaly and mild leukophilia and thrombocytosis that faithfully recapitulate human PV. A selective inhibitor of JAK2 prevents PV in rodents, thus offering proof of concept that JAK2 inhibitors can be effective treatment for PV. As it is currently used, this model is useful for testing inhibitors of JAK2 that are nonselective for wild type and mutant JAK2.

Initially, the rodent is dosed with both Epo and the test compound. The Epo binds to EpoR and induces a series of signaling events that result in homodimerization of EpoR, autophosphorylation of JAK2, transphosphorylation of EpoR, and phosphorylation of STAT5. The administration of the Epo to the rodent phenocopies all of the salient features of human PV. However, if the test compound is a selective inhibitor of JAK2, it will inhibit Epo-JAK2 signaling effectively preventing PV in the rodent.

The term “Epo”, as used herein refers to the hormone erythropoietin, which plays a crucial role in the regulation of red blood cell production and differentiation. In the current invention, the rodents can be dosed with endogenous human Epo, human recombinant Epo, rat Epo, and mouse Epo.

The structure of the endogenous human Epo molecule is characterized in Lai, et al., J Biol. Chem., 261(7): 3116-3121 (1986). Endogenous human Epo may contain from 4-14 sialic acid residues, therefore producing at least 11 different isoforms (Macdougall, Nephrol. Dial. Transplant. 17(Suppl. 5):66-70 (2002)). The rodent may be dosed with any isoform of endogenous Epo.

In addition to the endogenous Epo, the rodent may be dosed with any of the three forms of human recombinant Epo, which include “Epoetin alfa”, “Epoetin beta”, and “Darbepoetin alfa”. In one embodiment, the rodent may be dosed with Epoetin alfa, which structurally defined in Deechongkit, et al. (J. Pharma. Sciences, 95(9):1931-1943 (2006)). The Epoetin alfa utilized for dosing includes, but is not limited to, two commercially available products available under the trade names Epogen/Procrit® and Eprex®, which are produced by Amgen and Ortho Biotech Products, respectively.

In another embodiment, the rodent is dosed with the human recombinant “Epoetin beta”, which is structurally defined in Pronzato, et al. (Onco. Hemato., 58:46-52 (2006)). Epoetin beta utilized for dosing includes, but is not limited to, the commercially available product under the trade name NeoRecormon®, which is manufactured by Ortho Biotech Products.

In another embodiment, the rodent is dosed with the human recombinant Epo agent, “Darbepoetin alfa”, which is structurally defined in Macdougall, Nephrol. Dial. Transplant. 17(Suppl. 5):66-70 (2002). Darbepoetin alfa utilized for dosing includes, but is not limited to, the commercially available product, Aranesp®, which is manufactured by Amgen.

The term “test compound”, as used herein, refers to any compound, composition, polypeptide, protein, carbohydrate, lipid, lipoprotein, lipopolysaccharide, small molecule, or any combination therefore, that is to be screened for activity on JAK2.

The term “rodent” refers to any species of rat or mouse. In one embodiment, the rodent species utilized is the Balb/c strain. In another embodiment, the rodent utilized is from the C57B16 strain.

In one embodiment, the rat is dosed with the Epo and the test compound immediately. The term “immediately”, as used herein, indicates that the rodent is dosed with both the Epo and the test compound at the beginning of the experiment and the rodent is only dosed one time prior to sample collection.

In one embodiment, the rat is dosed with the Epo and the test compound daily. The term “daily”, as used herein, signifies that the rodent is dosed with both the Epo and the test compound one time a day for the length of the experiment. The length of the experiment may range between 1-28 days.

In one embodiment, the rat is dosed with the Epo and the test compound intermittently. The term “intermittently”, as used herein, refers to a daily dose of the test compound and a regulat time interval dose of Epo that is sufficient to result in commitment to red blood cell development or increased red cell mass. In one embodiment, the intermittent dosing for Epo was conducted every other day at the same time as the daily test compound dose was administered, for the length of the experiment. In a subclass, the intermittent dosing for Epo was conducted every other day for three days, at the same time as the daily test compound dose was administered.

The term “dosed” means any route of administration including, but not limited to, oral sample, subcutaneous injection, intravenous injection, and mini-pump.

After the rodent is dosed with the Epo and the test compound, a blood sample is collected up to 48 hours after the last dosing. In one embodiment, the biological sample is collected one hour after the dosing. In another embodiment, the biological sample is collected three hours after the dosing. Yet, in another embodiment, the biological sample is collected eight hours after the dosing.

The term “collected” refers to any means for obtaining a blood sample from a rodent. In one embodiment, the collection of blood is obtained while the animal is still alive retro-orbitally. In another embodiment, blood is collected while the animal is still alive through a tail bleed procedure. In another embodiment, the animal is euthanized and a blood sample is obtained through a cardiac puncture.

Finally, the collected blood samples are tested for phosphorylated STAT5 levels, concentration of the test compound, and Hematocrit levels. If the test compound inhibits JAK2 activity, then a decreased level of phosphorylated STAT5 will be detected. Additionally, the hematocrit level will be decreased, if the test compound is JAK2 inhibitor. The test compound concentration is ascertained, to determine the IC50 value for compounds that show JAK2 inhibitor activity.

In one embodiment, phosphorylated STAT5 levels are measured by X-MAP technology using Phosphorylated STAT 5A/B Beadmates (Millipore, Billerica, Mass.) on a BioPlex machine (Bio-Rad, Hercules, Calif.). In another embodiment, phosphorylated STAT5 levels are determined using a western blot. In another embodiment, phosphorylation of STAT5 is measured using flow cytometry.

Test compounds are measured directly by mass spectrometry. In one embodiment, the effect of test compound on JAK2 activity is measured by detecting the phosphorylation of JAK2 using antibodies.

In one embodiment, the hematocrit levels are ascertained by using an Advia 120 Hematology Analyzer (manufactured by Bayer/Siemens). In another embodiment, the hematocrit levels are determined using a Hemavet 960 (manufactured by Drew Scientific).

### EXAMPLE 1

Murine JAK2 Inhibition After Administration of Epo and Test Compound

Mice (4-6 weeks of age, C57B1/6 from Charles River Laboratories) are given a dose of Aranesp™ (10 Units/gram of body weight) by subcutaneous injection with a test compound (10-250 mg/kg of body weight by oral gavage). Retro-orbital blood was collected at 1 hour, 3 hours and 8 hours after dosing for determining the inhibitor concentration and phosphorylated STAT 5 levels. The blood sample was analyzed to determine the level of phosphorylated STAT5 by X-MAP technology using Phosphorylated STAT 5A/B Beadmates (Millipore, Billerica, Mass.) on a BioPlex machine (Bio-Rad, Hercules, Calif.). Levels of the test compound were quantified by comparing peak area ratio of the known compound and internal standard in samples to a standard curve in a LC/MS method using a positive MS/MS transition from m/z 363.9 to 199.6.

### EXAMPLE 2

Murine JAK2 Inhibition After Administration of Epo and Test Compounds: Efficacy Studies

Mice (4-6 weeks of age, C57B1/6 from Charles River Laboratories) were dosed with Aranesp® (10 Units/gram of body weight) by subcutaneous injection every other day for 7 days or plus a test compound (100 mg/kg by oral gavage) once a day for 7 days. On day 7, mice were euthanized and blood was collected via cardiac puncture. The blood was analyzed for Hematocrit, drug concentration and phosphorylated STAT 5 levels. Hematocrits were determined using a Hemavet 960 (Drew Scientific). Phosphorylated STAT5 levels were measured by X-MAP technology using Phosphorylated STAT 5A/B Beadmates (Millipore, Billerica, Mass.) on a BioPlex machine (Bio-Rad, Hercules, Calif.). Levels of test compound were quantified by comparing peak area ratio of the known compound and internal standard in samples to a standard curve in a LC/MS method using a positive MS/MS transition from m/z 363.9 to 199.6.

What is claimed is:

1. A method for identifying a test compound as an inhibitor of JAK2 activity comprising the steps of:
a) dosing a rodent with erythropoietin (Epo) and the test compound;
b) collecting blood samples after the dose is administered;
c) measuring phosphorylated STAT5 levels in the blood sample; and
d) determining JAK2 inhibitor levels in the blood samples.

2. The method of claim 1, wherein said Epo is selected from the group consisting of:
a) human recombinant Epo,
b) endogenous human Epo;
c) mouse Epo, and
d) rat Epo.

3. The method of claim 2, wherein said Epo is human recombinant Epo.

4. The method of claim 1, wherein said rodent is selected from the group consisting of:
a) Rat, and
b) Mouse.

5. The method of claim 1, wherein the blood samples may be collected at any time from 0 to 48 hours after dosing the rodent with the Epo and the test compound.

6. The method of claim 5, wherein the blood samples are collected at a time selected from the group consisting of:
a) 1 hour,
b) 3 hours, and
c) 8 hours.
after dosing of the rodent with the Epo and the test compound.

7. The method of claim 1, wherein said dosing of the rodent with Epo and the test compound occurs at the intervals selected from the group consisting of:
a) Immediately,
b) Daily, and
c) Intermittently.

8. The method of claim 1, wherein said collection of blood samples is obtained through the methods selected from the group consisting of
a) Retro-orbitally,
b) Cardiac puncture, and
c) Tail bleed.
